(19) United States
(12) Patent Application Publication
(10) Pub. No.: US 2024/0008797 A1
(43) Pub. Date: Jan. 11, 2024

(54) A TRIANGULAR WAVEFORM GENERATOR FOR A FAST-SCAN CYCLIC VOLTAMMETRY DEVICE

(71) Applicant: ISTANBUL MEDIPOL UNIVERSITESI, Istanbul (TR)

(72) Inventors: Mehmet KOCATÜRK, Beykoz, Istanbul (TR); Muhammad HAZIQ, Beykoz, Istanbul (TR); Raheel RIAZ, Beykoz, Istanbul (TR); Samet KOCATURK, Beykoz, Istanbul (TR)

(21) Appl. No.: 18/252,409

(22) PCT Filed: Nov. 16, 2021
(86) PCT No.: PCT/TR2021/051219

§ 371 Date: May 10, 2023

(30) Foreign Application Priority Data
Nov. 16, 2020 (TR) 2020/18279

(51) Int. Cl. A61B 5/00; A61B 5/25

(52) U.S. Cl. CPC A61B 5/4064; A61B 5/25

(57) ABSTRACT

Disclosed is a biomedical device capable of measuring dopamine by means of the FSCV technique.

### TECHNICAL FIELD OF THE INVENTION

The present invention relates to a novel biomedical device capable of measuring dopamine by implementing Fast-Scan Cyclic Voltammetry (FSCV) technique.

### STATE OF THE ART (PRIOR ART)

In the applications, patent documents, and systems available in the state of the art, it is observed that a digital-to-analog converter (DAC) is controlled by means of a microprocessor and the triangular wave required for measuring dopamine is generated by the DAC in the “Waveform Generator” component. This system, however, is quite costly.

In the prior art, a system implementing the FSCV technique was developed with the aim of measuring the neurotransmitter concentration in the brain. However, the voltammetric waveform required for the implementation of the FSCV technique was generated by means of a digital-to-analog converter (DAC) and a microprocessor (FIG. 2).

Digital-to-analog converters are purchased as ready-made integrated circuits from manufacturers in the prior art and ensured to generate the desired triangular voltammetric waveform by means of the commands given by the microprocessor.

### BRIEF DESCRIPTION OF THE INVENTION

The present invention, with the aim of overcoming all the disadvantages as mentioned above and to introduce further advantages into the relevant technical field, relates to a novel biomedical device that is capable of measuring dopamine by using FSCV technique.

Dopamine is a neurotransmitter released in the brain. It plays a vital role in motor control, motor learning, reinforcement learning, motivation, and decision making. Damage sustained by neurons that release dopamine leads to Parkinson's disease. Dopaminergic system dysfunctions are believed to be the cause of neuropsychiatric diseases such as schizophrenia, depression, and substance abuse. Therefore, understanding the physiology of the dopaminergic system is an important and active subject of research nowadays.

Measuring dopamine concentration changes in cerebral structures with high temporal resolution (at 100-millisecond intervals) is possible by using last-scan cyclic voltammetry (FSCV) technique. Today, this technique is used for measuring the changes that occur in dopamine concentration in the brain in experiments conducted with humans and animals. The technique has the potential to be used in neuro-prostheses for humans.

A microelectrode is positioned on a targeted area on the brain tissue in order to implement the FSCV technique. A triangular voltammetric wave is applied to said microelectrode and the current stemming from the reduction/oxidation of dopamine at the tip of the microelectrode is measured. Changes in dopamine concentration can be determined instantaneously based on the measured current value.

In the present invention, triangular voltammetric wave applied to the brain tissue is generated by using circuit components that are much more cost-efficient.

The present invention, in biomedical and neuroscience studies and in device implementations thereof, enables real-time measuring and monitoring the changes in dopamine concentration in the brain tissue.

The present invention may also be used in biomedical and other industrial devices for measuring the dopamine concentration.

### BRIEF DESCRIPTION OF THE DRAWINGS

Figures provided in order to ensure a better understanding of the triangular waveform generator for the fast-scan cyclic voltammetry device according to the present invention are disclosed below.

FIG. 1 is the schematic view of the Fast-Scan Cyclic Voltammetry Device (WE: Microelectrode used for recording dopamine; RE: Reference electrode; GND: Ground).

FIG. 2 is the “Waveform Generator” used in the present systems (PLL: Phase-Locked Loop; MCU: Microprocessor) (PRIOR ART). Waveform generators used in the present systems consist of a microprocessor (MCU) and a Digital-to-Analog Converter.

FIG. 3 is the “Waveform Generator” used in the present invention (PLL: Phase-Locked Loop; MCU: Microprocessor; U1-U3: comparator; U4-U5-U6-U7: op-amp; U2: optocoupler).

FIG. 4 is the triangular voltammetric wave generally used for measuring dopamine by means of fast-scan cyclic voltammetry.

FIG. 5 is the dopamine concentration measurement obtained in an experiment in which the present invention is used. In FIG. 5, the graph at the bottom shows the voltage value of the triangular wave generated by means of the “Waveform Generator” on the y-axis. The x-axis shows the time in seconds. The black arrow indicates the changes occurring in the Faraday current due to the dopamine concentration changes at the tip of the microelectrode positioned in the brain. This graph is called a voltammogram. In FIG. 5, the graph at the top shows the change in the dopamine concentration obtained with reference to said voltammogram. In said graph, y-axis shows dopamine concentration, while the x-axis shows time in seconds.

### REFERENCE NUMERALS

Parts/sections/elements are enumerated individually in order to provide a better understanding of the triangular waveform generator for the fast-scan cyclic voltammetry device developed by the present invention, and the equivalent of each number is given below.
1 Microelectrode (WE)
2 Reference Electrode (RE)
3 Waveform Generator
4 Phase-Locked Loop (PLL)
5 Current-to-Voltage Converter
6 Analog-to-Digital Converter and Data Logger
7 Microprocessor
8 Digital-to-Analog Converter (DAC)

### DETAILED DESCRIPTION OF THE INVENTION

In the detailed description provided herein, the present invention is described by means of examples that do not constitute any limiting effect and are intended only to provide better understanding of the subject matter.

The present invention relates to a fast-scan cyclic voltammetry device and a triangular waveform generator for said device. The present invention provides a novel component for the fast-scan cyclic voltammetry (FSCV) device. In the FSCV technique and a device thereof, the triangular voltammetric wave is generated in a specific voltage range and applied to a microelectrode positioned on the brain tissue. The triangular voltammetric wave is usually generated in a range between −0,4 Volts and +1,3 Volts. The initial voltage value (−0,4 Volts) and the peak voltage value (+1,3 Volts) of the triangular wave is selected according to the oxidation and reduction (0,7 Volts and −0,3 Volts respectively) voltages of dopamine, and in the way to scan these voltages. When the voltage value of the triangular wave applied to the microelectrode, beginning from −0,4 Volts and rising up to the peak voltage value of 1,3 Volts, reaches to 0,7 Volts, dopamine at the tip of the electrode is oxidized and transformed into dopamine-o-quinone. Decreasing from 1,3 Volts to −0,4 Volts, when it reaches to −0,3 Volts, it is reduced to dopamine-o-quinone and transforms back to dopamine. An exchange of electrons occurs between the microelectrode and dopamine during these chemical reactions. The current generated by the electron exchange is converted to voltage by means of the “Current-to-Voltage Converter” component of the FSCV device and is logged into the digital system (e.g., a computer) by means of its “Analog-to-Digital Converter and Data Logger” component. The triangular wave is applied every 100 milliseconds, thereby rendering it possible to measure the dopamine concentration 10 times in a second. The slope of the triangular wave is adjusted as 400 V/s, thus, taking measurements in every 100 milliseconds becomes possible. If the slope is at a lower value, then the microelectrode will have reduced precision, and consequently, measurements should be taken less frequently. Total length of the triangular wave applied as 400 V/s in a range between −0,4 Volts and +1,3 Volts corresponds to 8.5 milliseconds. The voltage applied to the microelectrode is maintained at −0,4 Volts when the triangular wave is not applied. Thus, a larger number of dopamine molecules are gathered around the microelectrode, thereby improving the dopamine measurement precision of the microelectrode. FIG. 4 illustrates the triangular waveform.

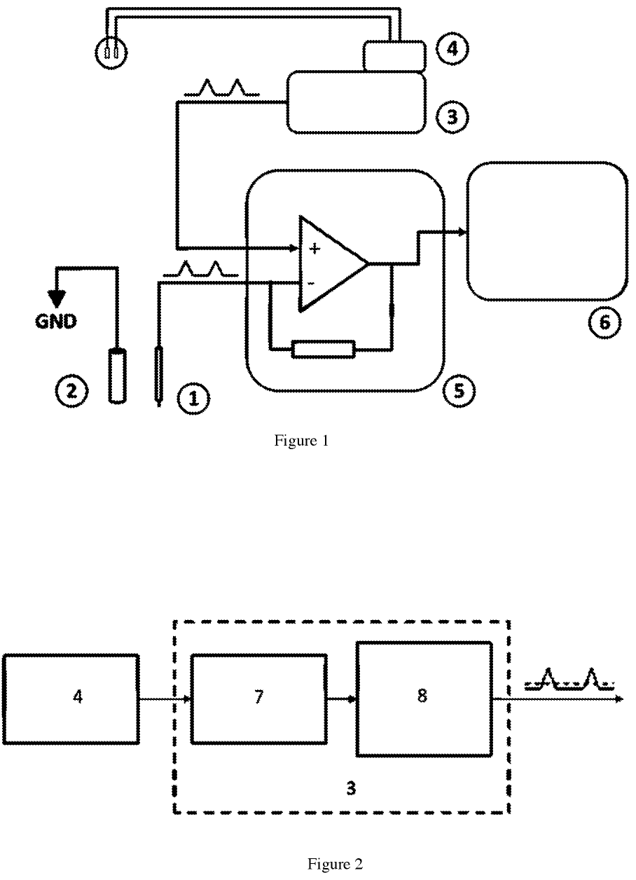

FIG. 1 illustrates the fast-scan cyclic voltammetry (FSCV) device and the components thereof. The FSCV device is comprised of 6 components. 1-Microelectrode (WE), 2-Reference Electrode (RE), 3-Waveform Generator, 4-Phase-Locked Loop (PLL), 5-Current-to-Voltage Converter, 6-Analog-to-Digital Converter and Data Logger. FIG. 1 illustrates a mains power socket as well.

The present invention implements the last-scan cyclic voltammetry (FSCV)′ technique, which is an electrochemical technique. The present invention measures the changes occurring in dopamine concentration in the brain by implementing said technique.

In the present invention, the triangular wave sign required for implementing the FSCV technique is generated by means of a microprocessor that generates a square wave signal, and an analog circuit that takes the integral of said square wave signal. Thereby, system components can be much more cost-efficient.

A microelectrode (1) is implanted into the brain tissue, while a reference electrode (2) is implanted into brain or into another tissue. Waveform generator (3) generates a triangular wave. The waveform generator (3) transmits this waveform to the current-to-voltage converter (5) in order to be applied to the microelectrode (1). The current-to-voltage converter applies the triangular waveform to the microelectrode (1). A current is generated on the microelectrode (1) as a result of applying the triangular wave. In FSCV technique, this current is called a background current. In addition to the generated background current, dopamine at the tip of the microelectrode is reduced and oxidized upon applying the triangular waveform to the microelectrode. A Faraday current that is in direct proportion to the dopamine concentration is generated on the microelectrode, in addition to the background current, as a result of this chemical reaction. The background current and the Faraday current are jointly converted to voltage by means of the current-to-voltage converter (5) and transmitted to the analog-to-digital converter and data logger (6). “Analog-to-Digital Converter and Data Logger (6)” records the background current and the Faraday current values converted to voltage with high frequency (e.g., 100 KHz). After the recording operation is complete, the background current is subtracted from the recorded current values mathematically through subtraction and the Faraday current is obtained as a result of this operation. The obtained Faraday current corresponds to the dopamine concentration in the medium. Dopamine concentration is determined based on the Faraday current by using computational methods.

The phase-locked loop (PLL) (4) in the FSCV device is used for removing the noise interference originating from the mains power grid. The waveform generator (3) detects the signal phase of the mains power grid by means of the phase-locked loop (PLL) (4) and generates the triangular waves such that said waves are locked to this signal phase. Thus, noise interferences originating from the mains power grid during the measuring operations are removed performing a mathematical subtraction after the recording operation is complete.

In the present invention, the component marked with the reference number of 3 (Waveform Generator) as illustrated in FIG. 1 is novel. In the prior art, the “Waveform Generator” consists of at least one digital-to-analog converter (DAC) and at least one microprocessor (FIG. 2).

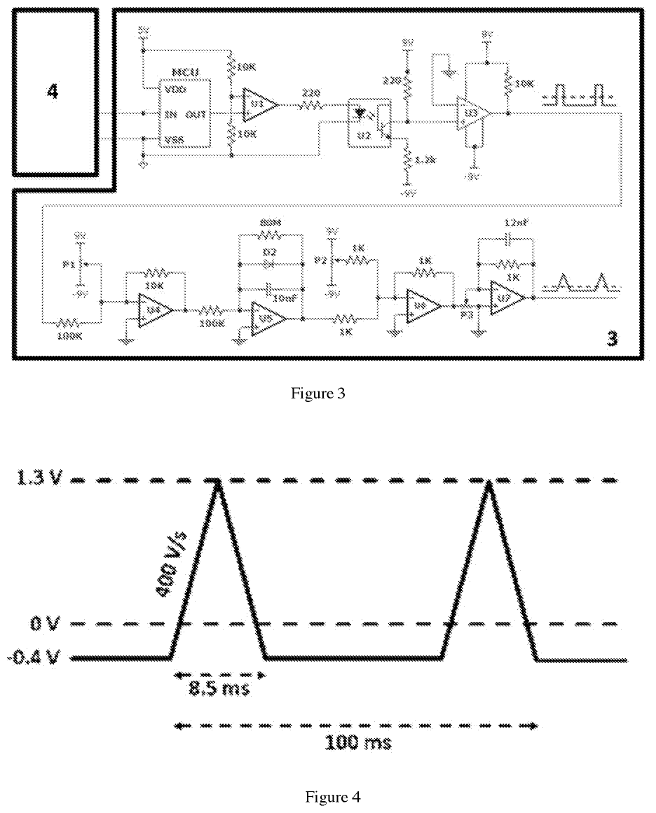

In the present invention, the circuit illustrated in FIG. 3 is proposed as the waveform generator. This design offers 2 advantages: 1-Circuit components used therein are much more cost-efficient. 2-The electrical noise generated by the microprocessor is prevented from causing interference in the triangular wave by using an optocoupler.

Respective details regarding the operation of the present invention are disclosed below.

FIG. 3 illustrates the circuit diagram of the present invention. In the present invention, the “Waveform Generator” comprises a microprocessor (MCU). The microprocessor, based on the input provided by the PLL (4), generates pulse signals, phase of which is locked to the signal of the mains power grid. Comparator integrated circuit number U1 performs a voltage comparison and applies the pulse signal generated to the input of the optocoupler (component number U2 shown in FIG. 3). Comparator integrated circuit number U3 performs a voltage comparison at the output of the optocoupler and generates a pulse signal. An operational amplifier (opamp) integrated circuit (U4) is used for decreasing the amplitude and shifting of the pulse signal generated at the output of the optocoupler. Subsequently, the integral of the pulse signal is taken by means of an integrating circuit (U5), thereby converting the pulse signal to triangular wave. D2 diode plays a crucial role here; it prevents the U5 opamp of from reaching saturated state and ensures that the operation of taking integral is performed smoothly. The voltage of the triangular wave is shifted upwards or downwards by means of the opamp number U6. Finally, the peak-to-peak amplitude of the triangular wave is adjusted by means of opamp number U7. This design allows for generating the waveform which is commonly used for measuring dopamine as illustrated in FIG. 4.

In the present invention, the width and the generation frequency of the pulse signals generated by the microprocessor may be changed as desired. Thus, the width and the generation period of the triangular wave can be adjusted. In FIG. 4, these parameters are indicated as 8.5 milliseconds and 100 milliseconds, respectively. Initial and peak voltages of the triangular wave may also be adjusted as desired by using the components shown in FIG. 3. In FIG. 4, these parameters are indicated as −0.4V and 1.3V, respectively.

Said indicated parameters of the triangular wave may be adjusted as desired in the present invention.

Different initial and peak voltages, different triangular wave widths and different triangular wave generation periods may be achieved for different applications by means of the present invention.

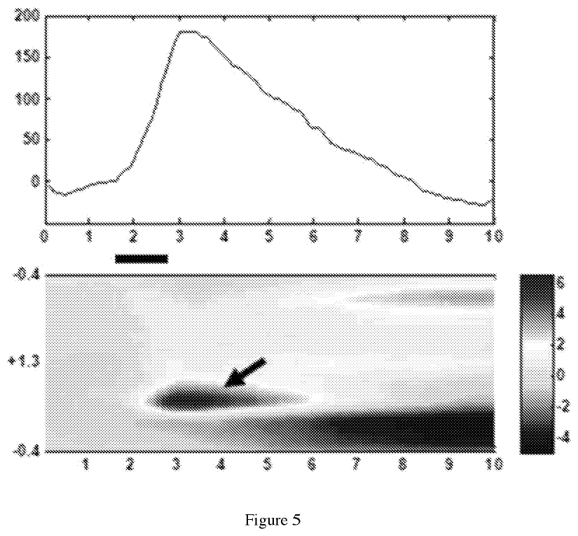

FIG. 5 illustrates the measurement of dopamine concentration obtained in an experiment in which the present invention is used. The black horizontal line between two graphs indicates the moment in which dopamine neurons are stimulated. The graph at the bottom (applied voltage-time graph) shows the Faraday current generated as a result of reducing/oxidizing dopamine by means of the applied triangular wave. The Faraday current is indicated by the black arrow.

The present invention, in line with the detailed information provided above, is a device that enables measuring changes occurring in the dopamine concentration in the brain, characterized in that it is a triangular wave generator that enables detecting the signal phase of the mains power grid by means of phase-locked loop (PLL), allows generating triangular waves such that the waves are locked to the phase of said signal, and transmits the said waveform to current-to-voltage converter to be applied to microelectrode.

Said triangular wave generator is characterized in that it comprises;
A microprocessor (MCU) (7) that enables generating pulse signals, phases of which are locked to the signal of the mains power grid, based on the input provided by the phase-locked loop (PLL) (4),
Comparator integrated circuit (U1) that enables making a comparison for the pulse signals generated by the microprocessor, and allows applying the generated pulse signal to the input of the optocoupler (component number U2 shown in FIG. 3),
An optocoupler (U2) that ensures electrical insulation between the microprocessor (7) and the waveform generator (3), hereby preventing electrical noise which originates from the microprocessor to cause interference in the generated triangular wave,
Comparator integrated circuit (U3) that enables generating pulse signal at the output of the optocoupler (U2),
Operational amplifier (opamp) integrated circuit (U4) that enables decreasing the amplitude and shifting of the generated pulse signal,
U5 opamp that enables taking the integral of the pulse signal for converting the pulse signal to a triangular wave,
A diode (D2) that prevents the opamp (U5), which enables taking integral, from reaching saturated state and ensures that the operation of taking integral is performed smoothly,
U6 opamp that enables shifting the voltage of the triangular wave upwards or downwards,
U7 opamp that allows adjusting the peak-to-peak amplitude of the triangular wave.

The device that enables measuring the changes occurring in the dopamine concentration in the brain and comprises the elements of; a microelectrode (1) that is implanted into the brain tissue; a reference electrode (2) that is implanted into the brain or into another tissue; a phase-locked loop (PLL) (4) that is provided inside the fast-scan cyclic voltammetry (FSCV) device and that is used for removing noise interferences originating from the mains power grid; a current-to-voltage converter (5) that enables applying the triangular waveform to the microelectrode (1); and an analog-to-digital converter and data logger (6) that enables recording the background current and the Faraday current values converted to voltage; comprises the triangular wave generator mentioned above. Said device is a fast-scan cyclic voltammetry device.

A method for operating the device that enables measuring the changes occurring in the dopamine concentration in the brain, characterized in that, said method comprises the process steps of;
Implanting the microelectrode (1) into the brain tissue, and the reference electrode (2) into the brain or into another tissue,
Generating a triangular wave by means of the waveform generator (3),
Transmitting the waveform to the current-to-voltage converter (5) by means of the waveform generator (3) in order for applying said waveform to the microelectrode (1),
The current-to-voltage converter (5) applying the triangular waveform to the microelectrode (1),
Obtaining a background current on the microelectrode (1) upon applying the triangular wave,
Reducing and oxidizing the dopamine at the tip of the microelectrode by applying the triangular wave to the microelectrode in addition to the obtained background current,
Obtaining a Faraday current that is in direct proportion to the dopamine concentration on the microelectrode in addition to the background current as a result of this chemical reaction,
Converting the obtained background current and the Faraday current jointly to voltage by means of the current-to-voltage converter (5) and transmitting to the analog-to-digital converter and data logger (6),
The analog-to-digital converter and data logger (6) recording the values of the background current and the Faraday current converted to voltage,
Subtracting the background current from the recorded current values mathematically through subtraction after the recording operation is complete, and obtaining the Faraday current that corresponds to the dopamine concentration in the medium as a result of this operation,
Determining the dopamine concentration by using the linear relationship between the obtained Faraday current and the dopamine concentration in the medium, and by calculating the ratio.

1. A device that enables measuring changes occurring in the dopamine concentration in the brain, the device comprising a triangular wave generator that detects a signal phase of a mains power grid by means of a phase-locked loop (PLL), and generates triangular waves such that the waves are locked to the phase of said signal, and transmits said waveform to a current-to-voltage converter to be applied to a microelectrode.

2. A device according to claim 1, wherein the triangular wave generator comprises;
a microprocessor that enables generating pulse signals, phases of which are locked to the signal of the mains power grid, based on the input provided by the phase-locked loop (PLL),
a comparator integrated circuit that enables making a comparison for the pulse signals generated by the microprocessor, and allows applying the generated pulse signal to the input of the optocoupler,
an optocoupler that ensures electrical insulation between the microprocessor and the waveform generator, thereby preventing electrical noise which originates from the microprocessor to cause interference in the generated triangular wave,
a comparator integrated circuit that enables generating pulse signal at the output of the optocoupler,
an operational amplifier (opamp) integrated circuit that enables decreasing the amplitude and shifting of the generated pulse signal,
an opamp that enables taking the integral of the pulse signal for converting the pulse signal to a triangular wave,
a diode that prevents the opamp that enables taking integral from reaching saturated state and ensures that the operation of taking integral is performed smoothly,
an opamp that enables shifting the voltage of the triangular wave upwards or downwards, and
an opamp that allows adjusting the peak-to-peak amplitude of the triangular wave.

3. A device comprising a microelectrode that is implanted into the brain tissue, a reference electrode that is implanted into the brain or into another tissue, a phase-locked loop (PLL) that is provided inside a fast-scan cyclic voltammetry (FSCV) device and that is used for removing noise interferences originating from the mains power grid, a current-to-voltage converter that enables applying the triangular waveform to the microelectrode, an analog-to-digital converter and data logger that enables recording the background current and the Faraday current values converted to voltage, wherein it comprises the triangular wave generator according to claim 2.

4. A method for operating the device according to claim 3, comprising the process steps of:
implanting the microelectrode into the brain tissue, and the reference electrode into the brain or into another tissue,
generating a triangular wave by means of the waveform generator,
transmitting the waveform to the current-to-voltage converter by means of the waveform generator in order for applying said waveform to the microelectrode,
the current-to-voltage converter applying the triangular waveform to the microelectrode,
obtaining a background current on the microelectrode upon applying the triangular wave,
reducing and oxidizing the dopamine at the tip of the microelectrode by applying the triangular wave to the microelectrode in addition to the obtained background current,
obtaining a Faraday current that is in direct proportion to the dopamine concentration on the microelectrode in addition to the background current as a result of this chemical reaction,
converting the obtained background current and the Faraday current jointly to voltage by means of the current-to-voltage converter and transmitting to the analog-to-digital converter and data logger,
the analog-to-digital converter and data logger recording the values of the background current and the Faraday current converted to voltage,
subtracting the background current from the recorded current values mathematically through subtraction after the recording operation is complete, and obtaining the Faraday current that corresponds to the dopamine concentration in the medium as a result of this operation, and
determining the dopamine concentration by using the linear relationship between the obtained Faraday current and the dopamine concentration in the medium, and by calculating the ratio.

5. A device according to claim 1, wherein the device is a fast-scan cyclic voltammetry device.

6. A device according to claim 3, wherein the device is a fast-scan cyclic voltammetry device.
